(19) United States
(12) Patent Application Publication
(10) Pub. No.: US 2026/0325329 A1
(43) Pub. Date: Oct. 22, 2026

(54) STRUCTURAL ARRANGEMENT IN A BALLOON CATHETER DEVICE AND DEDICATED BIFURCATION STENT

(71) Applicant: Luiz Alberto Rolla Maneschy, Belém (BR)

(72) Inventor: Luiz Alberto Rolla Maneschy, Belém (BR)

(21) Appl. No.: 19/107,628

(22) PCT Filed: Apr. 20, 2023
(86) PCT No.: PCT/BR2023/050124

§ 371 Date: Feb. 28, 2025

(30) Foreign Application Priority Data
Aug. 31, 2022 (BR) 2020220175344

(51) Int. Cl. A61F 2/954; A61F 2/958; A61M 25/09

(52) U.S. Cl. CPC A61F 2/954; A61F 2/958; A61F 2250/0098; A61M 25/09

(57) ABSTRACT

The present utility model patent application belongs to the field of surgical accessories and refers more specifically to a balloon catheter used in vessels with bifurcation, which notably has a stent, which facilitates the delivery of the stent by placing the angle in the respective bifurcated region, having a variable angular cut from 10° to 80° in the proximal portion, allowing the stent to completely cover the atherosclerotic plaque of the side branch, substantially reducing the possibility of protruding into the main branch; the congregation of the new balloon catheter and stent model no longer demands the need to use complex techniques, since it simplifies the implant procedure, allowing the correct application of the prosthesis at its respective angle; the improvement solves the treatment in the application in bifurcation lesions so that it can fully cover the vessel(s) at the level of the carina, can be used to treat lesions in one branch of a bifurcation, while preserving access to the other branch for future treatment and also reducing the risk of restenosis, thrombosis and preserving blood flow from the side/main branches.

### TECHNICAL FIELD OF THE INVENTION

The present utility model patent application belongs to the field of surgical accessories and refers more specifically to a balloon catheter for use in vessels with bifurcation, which notably has a drug-eluted stent (drug-eluting stent), which aims to facilitate the delivery of the stent through the perfect placement of the angle in the respective bifurcated region, having a variable angular cut from 10 ° to 80 ° in the proximal portion, allowing the stent to completely cover the atherosclerotic plaque of the side branch, substantially reducing the possibility of protruding to the main branch.

The congregation of the new balloon catheter and stent model no longer requires the need to use complex techniques, since it simplifies the implant procedure, allowing the correct application of the prosthesis at its respective angle.

Such an enhancement solves the treatment on application in bifurcation lesions so that it can fully cover the vessel(s) at the level of the carina, can be used to treat lesions in one branch of a bifurcation, while preserving access to the other branch for future treatment and also reducing the risk of restenosis, thrombosis and preserving blood flow from the side/main branches.

### TECHNICAL BACKGROUND

Stents are commonly used to be implanted within an artery, vein, or other tubular organ, for treatment of occlusions, stenoses, or aneurysms of a vessel to reinforce the vessel wall or expand it. Stents are also used to treat dissections in the walls of blood vessels, caused by dilation of the balloon catheter, improving the results of the procedure, preventing elastic retraction and remodeling of the vessel wall, and rupture of a vulnerable plaque.

### ANALYSIS OF THE STATE OF THE ART

In research carried out in specialized databases, documents referring to stents were found, such as document No. BR 202019018280-1, which is the present utility model patent, of a new constructive arrangement introduced in a stent release device, belonging to the area of medicine, more particularly it is a device for the release of stent for endovascular treatment, in particular in regions where there is a need for greater precision at the limit point of stent release, avoiding, for example, the obstruction of the bifurcation of the iliac arteries. The stent release device is formed by a catheter (1), provided at the end of a tip (11) with a conical trunk body, preferably in silicone, atraumatic and flexible, and through-hole (12), which is surrounded by a proximal outer cover (2) and also at the inner end a compressed stent (3) is provided, the tip (11) being provided with a tubular extension (111), in a single body, forming a housing (112) for the stent (3), compressed, the tubular extension (111) being of the same length as the stent (3) and separated from the outer cover (2), forming the dividing region (4) of the beginning of displacement of the stent (3), coincident with the limit point (PL).

The document No. PI 0616331-9 which deals with a “BIFURCATED BALLOON AND STENT”, the present invention relates to a stent (22) for the treatment of a vascular bifurcation includes a distal element (40) and a proximal element (42), the distal element and the proximal element comprising tubular bodies, the distal element comprising first struts (64, 66) oriented in the proximal direction and the proximal element including second struts (54, 56) oriented in the distal direction, such that at least one of the first struts is connected to at least one of the second struts on a first side of the stent (46), while on a second side (48) of the stent, opposite to the first side, one or more of the first struts are not connected to any of the second struts and one or more of the second struts are not connected to any of the first struts.

Document No. PI 0415518-1 dealing with a “CATHETER SYSTEM FOR INSTALLING A STENT IN BIFURCATED VESSELS”, the present patent application relates to a catheter system and a method for installing a stent in a vessel at the level of a bifurcation or a branch of the vessel; the catheter system comprises a first balloon catheter, a second balloon catheter and a release linking device for servicing the first and second balloon catheters disposed side-by-side or aligned relative to each other along a longitudinal axis; the linking system allows advancing the catheter system from one unit and helps prevent premature or inadvertent stent displacement of the catheters, however it is releasable such that one or both of the balloon catheters can be released from the linking device and be maneuvered separately from the rest of the catheter system when desired; the method utilizes the catheter system described for the installation in a vessel bifurcation using a modified ‘Kissing Ballons’ technique.

Although the most varied models of stents are available on the market, they are manufactured in a straight tubular configuration and the use of these stents to treat vessels at bifurcation points of a vessel can create a risk of compromising the patency of the main vessel or its branch, also limiting the eventual release of another stent in the side branch if the result of stent treatment in the main vessel is suboptimal.

Suboptimal results may occur due to various mechanisms such as atherosclerotic plaque displacement, vessel spasm, dissection, thrombosis, and embolism. In addition, the use of conventional stents to treat bifurcations typically requires the use of multiple stents to completely cover the bifurcation of the vessels, which can lead to overlapping of stents or, conversely, gaps between the stents that prevent obtaining adequate structural formation.

The existence of the angle at the level of the bifurcation prevents the stent of the side branch from being perfectly released, occurring either incomplete coverage of the branch or the release of the stent in the lumen of the main vessel. In the treatment of bifurcation lesions, using the available conventional stents, it is important to cover the origin of the side branch, otherwise this area will be prone to restenosis. In order to cover the origin of the side branch, conventional stents protrude into the lumen of the main branch, which can cause thrombosis and compromise blood flow. Another frequent complication that occurs, in a bifurcation of a vessel treated with the stent in the main branch, is the narrowing or occlusion of the origin of the side branch. To solve this, it is currently necessary to release another stent in the side branch, but usually this stent does not cover the origin of the bifurcation, thus making future access to this branch difficult or impossible.

Thus, it is a fact that the documents cited in the above paragraphs, despite belonging to the same field of application, do not present any of the characteristics of the object now improved, thus ensuring that it meets the legal requirements of patentability.

### BRIEF DESCRIPTION OF THE INVENTION

Taking into account all the problems that were presented in the state of the art, the inventor, who has notorious knowledge about the sector, created and developed the aforementioned “STRUCTURAL ARRANGEMENT IN A BALLOON CATHETER DEVICE AND DEDICATED STENT FOR BIFURCATION”.

The objective of the present invention is to present a balloon catheter, having a stent, which is intended to facilitate the delivery of the stent through the perfect placement of the angle in the respective bifurcated region, having an angular cut (variable from 10 ° to 80 °) in the proximal portion, allowing the stent to completely cover the atherosclerotic plaque of the side branch, substantially minimizing the possibility of protruding into the main branch.

Angioplasty of lesions in bifurcations is performed using various techniques such as T stenting, Culotte, Crush, V stenting. Additional techniques are used such as kissing balloon and kissing stenting, where two balloons or two stents are inflated simultaneously in the main vessel and in the side branch, in order to optimize the final result. However, the congregation of the new balloon catheter and stent model will no longer require the use of some of the aforementioned techniques, since it will simplify the implant procedure, allowing the correct application of the prosthesis at its respective angle.

Such an enhancement solves the treatment on application in bifurcation lesions so that it can fully cover the vessel(s) at the level of the carina, can be used to treat lesions in one branch of a bifurcation, while preserving access to the other branch for future treatment and also reducing the risk of restenosis, thrombosis and preserving blood flow from the side/main branches.

The present invention does not only take into account the mechanical and functional qualities, but also the form, arrangement and strategic location of its parts and components that, correctly positioned, have brought an increase in efficiency without incurring any burden to users.

### DESCRIPTION OF DRAWINGS

To complement the present description in order to obtain a better understanding of the features of the present invention and according to a preferred practical realization thereof, the attached description is accompanied by a set of drawings, where, in an exemplary, although not limiting manner, its operation was represented:

FIG. 1 illustrates an image of the stent according to the invention, demonstrating the angled cut from 10 ° to 80 °;

FIG. 2 discloses a side view of the present balloon catheter with the first guide that faces the side branch and the second guide that is located on the main branch;

FIG. 3 shows a side view of the stent mounted on the balloon catheter, where we can notice the first guide directed to the side branch and the second guide on the main branch.

FIG. 4 presents an image of the balloon catheter simulating the placement of the stent side branch of the bifurcation;

FIG. 5 displays an image of the stent being deployed;

FIG. 6 illustrates a perspective view of the stent deployed at the bifurcation site with the angulation compatible with the angle formed between the side branch and the main branch; and

FIG. 7 shows another perspective view of the stent deployed at the bifurcation site with the angulation compatible with the angle formed between the side branch and the main branch.

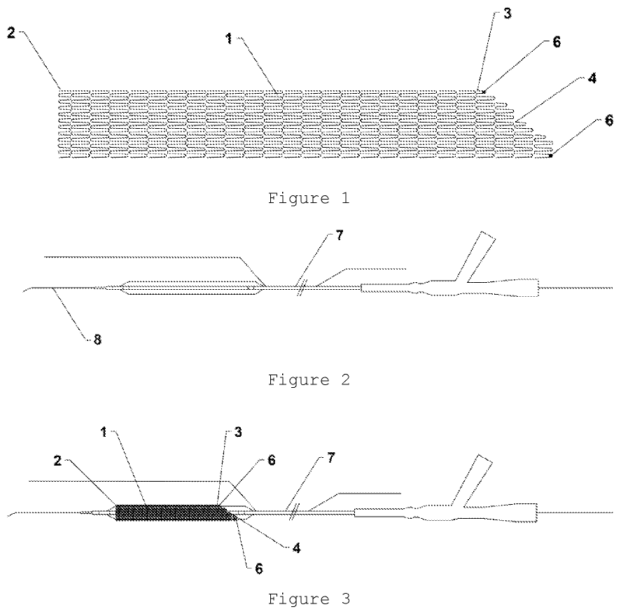

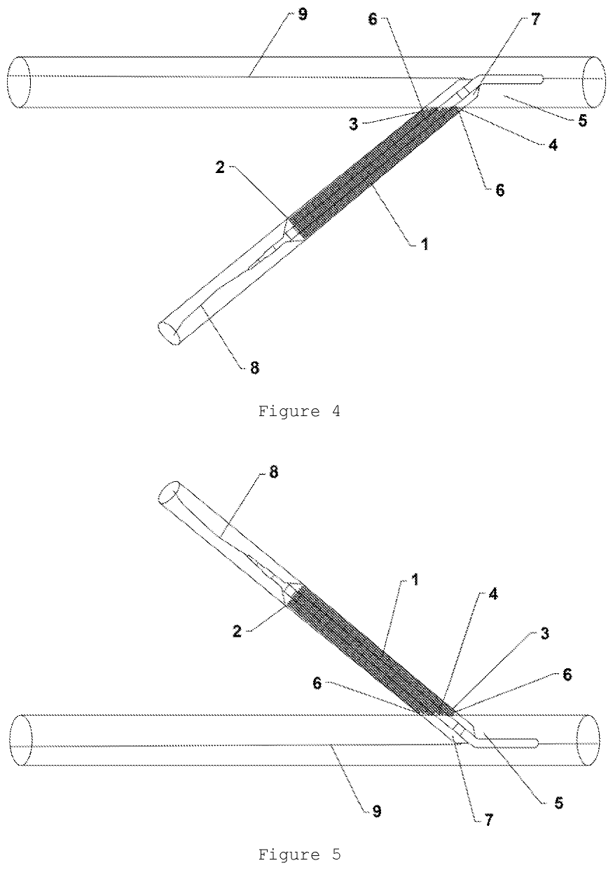

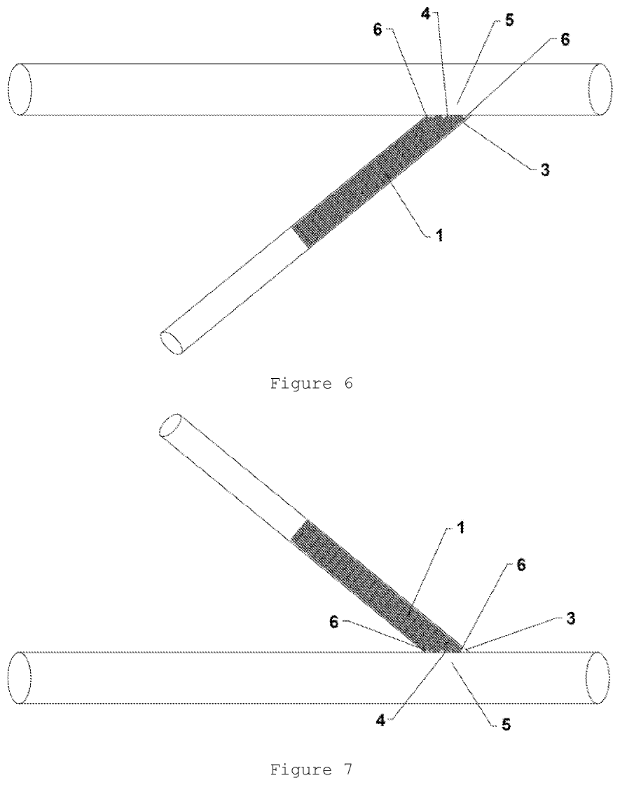

### DETAILED DESCRIPTION OF THE INVENTION

With reference to the illustrated drawings, the present utility model patent refers to the “STRUCTURAL ARRANGEMENT IN A BALLOON CATHETER DEVICE AND DEDICATED STENT FOR BIFURCATION”, more precisely it conceives a stent (1) dedicated for use in vessels with bifurcation and designed to be released only in the side branch, which is manufactured from a single tubular structure, being configured for radial expansion.

The distal portion (2) of the stent (1) is formed in the conventional way, and the proximal portion (3) presents a cut (4) with angles varying from 10 ° to 80 °, so that the stent can completely cover the atherosclerotic plaque (5) of the side branch, and not have the possibility of protruding into the main branch.

The present invention has in its proximal portion (3) two radiopaque marks (6) that will indicate the exact location that will be released, providing a clear visualization of the implant angulation.

The solution provides a balloon catheter (7), in which the stent (1) is crimped, which presents a specific configuration for release, enabling the delivery of the implant at the respective angulation pertinent to the side branch of the bifurcation.

The first guide wire (8) enters the distal portion of the balloon catheter (7) and exits the proximal portion (3) by which the balloon catheter (7) will follow with the stent (1) to be positioned on the side branch over the lesion. The other guide wire (9) enters the proximal portion of the balloon, externalizes into the body of the balloon catheter (7) and positions itself in the main branch. This new configuration of the balloon catheter (7) with two guide wires (8) and (9), allows the stent (1) with the angled cut (4) to be delivered correctly and compatible with the angle of the side branch, covering the entire plaque (5) without invading the main branch and allowing blood flow avoiding the risk of restenosis.

It is certain that when the present invention is put into practice, modifications may be introduced with regard to certain details of construction and form, without this implying departing from the fundamental principles that are clearly substantiated in the set of claims, thus being understood that the terminology employed was not intended as a limitation.

1. “CONSTRUCTIVE ARRANGEMENT APPLIED IN BALLOON CATHETER DEVICE and DEDICATED STENT FOR BIFURCATION”, conceives a stent (1) dedicated for use in vessels with bifurcation and designed to be released only in the side branch, which is manufactured from a single tubular structure, being configured for radial expansion, having a conventional shape in its distal portion (2);
characterized in that the proximal portion (3) presents a cut (4) with angles varying from 10 ° to 80 °, completely covering the atherosclerotic plaque (5) of the side branch; having in its proximal portion (3) two radiopaque marks (6) indicating the exact location to be released, visualizing the angulation of the implant; the stent (1) being crimped to a balloon catheter (7) manufactured in the over-the-wire mode; presenting a specific configuration for release, enabling the delivery of the implant at the respective angulation pertinent to the side branch of the bifurcation; having a first guide wire (8) which enters the distal portion of the balloon catheter (7) and exits in the proximal portion (3) following the balloon catheter (7) with the stent (1) to be positioned in the side branch over the lesion; containing a second guide wire (9), which enters the proximal portion of the balloon, externalizing itself in the body of the balloon catheter (7) and positioning itself in the main branch.

2. A medical device comprising:
a single-piece, radially expandable, tubular stent comprising:
a distal end substantially perpendicular to a length of the stent;
a proximal end sloped relative to the length of the stent; and
at least one radio-opaque marker positioned near the proximal end.

3. The medical device of claim 2, further comprising a ballon catheter, wherein the stent is secured to the balloon catheter.

4. The medical device of claim 3, wherein the stent is crimped to the balloon catheter.

5. The medical device of claim 2, further comprising a first guidewire extending from an end of the ballon catheter proximate the distal end of the stent and defining a first path.

6. The medical device of claim 5, wherein the first path extends lengthwise through the stent.

7. The medical device of claim 5, further comprising a second guidewire that follows a second path different than the first path.

8. The medical device of claim 7, wherein a first portion of the second path is internal to the balloon catheter and a second portion of the second path is external to the balloon catheter.

9. The medical device of claim 2, wherein the proximal end slopes at an angle between 10 and 80 degrees relative to the length of the stent.

10. The medical device of claim 2, wherein the at least one radio-opaque marker positioned near the proximal end comprises:
a first radio-opaque marker positioned at a first location on the proximal end closest to the distal end; and
a second radio-opaque marker positioned at a second location on the proximal end farthest from the distal end.

11. The medical device of claim 2, wherein the proximal end defines a perimeter, and the at least one radio-opaque marker positioned near the proximal end comprises:
a first radio-opaque marker positioned on the perimeter; and
a second radio-opaque marker positioned on the perimeter opposite the first radio-opaque marker.

12. A medical device comprising:
a catheter;
a selectively inflatable balloon secured to the catheter;
a first guidewire extending through at least a portion of the catheter;
a selectively expandable stent removably secured to the catheter, the stent comprising:
a tubular body defining a central lumen;
a first end substantially perpendicular to the tubular body and defining a substantially circular perimeter; and
a second end opposite the first end and sloped relative to the tubular body, the second end defining a substantially ovular perimeter.

13. The medical device of claim 12, wherein the stent further comprises at least one radio-opaque marker.

14. The medical device of claim 13, wherein the at least one radio-opaque marker comprises two radio-opaque markers, each positioned at a vertex of the substantially ovular perimeter.

15. The medical device of claim 12, wherein the second end slopes at an angle between 10 and 80 degrees relative to the tubular body.

16. The medical device of claim 12, further comprising a second guidewire extending through at least a portion of the catheter.

17. The medical device of claim 16, wherein the second guidewire extends through the central lumen.

18. The medical device of claim 16, wherein the first guidewire is configured to extend through a lumen of a principal blood vessel and the second guidewire is configured to extend through a lumen of a secondary blood vessel that branches off of the principal blood vessel.

19. The medical device of claim 12, wherein the selectively expandable stent is configured to be positioned within a secondary blood vessel that branches off of a principal blood vessel, with the proximal end proximate the principal blood vessel and the stent covering substantially all of an interior wall of the secondary blood vessel proximate the principal blood vessel.

20. The medical device of claim 19, wherein the selectively expandable stent is configured to be radially expanded and detached from the catheter when so positioned.
